(19) United States
(12) Patent Application Publication
(10) Pub. No.: US 2020/0352464 A1
(43) Pub. Date: Nov. 12, 2020

(54) SYNCHRONIZING NEUROELECTRIC MEASUREMENTS WITH DIAGNOSTIC CONTENT PRESENTATION

(71) Applicant: X Development LLC, Mountain View, CA (US)

(72) Inventors: Sarah Ann Laszlo, Mountain View, CA (US); Nina Thigpen, Sunnyvale, CA (US); Vladimir Miskovic, Binghamton, NY (US); Yvonne Yip, San Francisco, CA (US)

(21) Appl. No.: 16/871,942

(22) Filed: May 11, 2020

Related U.S. Application Data
(60) Provisional application No. 62/846,256, filed on May 10, 2019.

(51) Int. Cl. A61B 5/04; A61B 5/0476; A61B 5/00

(52) U.S. Cl. CPC A61B 5/04012; A61B 5/04004; A61B 5/0476; A61B 2562/02; A61B 5/7285; A61B 5/7435; A61B 5/7267

(57) ABSTRACT

Methods, systems, and apparatus, including computer programs encoded on a computer storage medium, for synchronizing neuroelectric measurements with diagnostic content presentation by performing actions that include causing a presentation system to present diagnostic content a user, where the diagnostic content includes: a first content frame prompting the user to perform a physical task, a second content frame including electroencephalogram (EEG) synchronization content, the second content frame sequentially following the first content frame, and a third content frame indicating an outcome of the physical task, the third content frame sequentially following the second content frame. The actions include obtaining, from a brainwave sensor, EEG signals of the user during presentation of the diagnostic content. The actions include providing the EEG signals as input features to a machine learning model that is trained to predict a psychological state of the user based on the EEG signals.

### CROSS-REFERENCE TO RELATED APPLICATION

This application claims the benefit under 35 U.S.C. § 119(e) of U.S. Patent Application No. 62/846,256, entitled “Synchronizing Neuroelectric Measurements With Diagnostic Content Presentation,” filed May 10, 2019, which is incorporated herein by reference in its entirety.

### TECHNICAL FIELD

This disclosure generally relates to psychophysiological measurement systems. More particularly, the disclosure relates to processes for improving the data acquisition process for psychophysiological measurement systems.

### BACKGROUND

Psychophysiological measurement systems, such as electroencephalogram (EEG) systems, are used for research and diagnostic purposes. Content is presented to study participants and/or patients while psychophysiological measurements are conducted, e.g., to trigger various psychophysiological responses by the study participants and/or patients.

### SUMMARY

In general, innovative aspects of the subject matter described in this specification can be embodied in methods that include the actions of causing a presentation system to present diagnostic content a user, where the diagnostic content includes: a first content frame prompting the user to perform a physical task, a second content frame including electroencephalogram (EEG) synchronization content, the second content frame sequentially following the first content frame, and a third content frame indicating an outcome of the physical task, the third content frame sequentially following the second content frame. The actions include obtaining, from a brainwave sensor, EEG signals of the user during presentation of the diagnostic content. The actions include providing the EEG signals as input features to a machine learning model that is trained to predict a psychological state of the user based on the EEG signals. Other implementations of this aspect include corresponding systems, apparatus, and computer programs, configured to perform the actions of the methods, encoded on computer storage devices. These and other implementations can each optionally include one or more of the following features.

In some implementations, the second content frame includes EEG synchronization content configured to establish a baseline EEG signal. In some implementations, the EEG synchronization content includes fixation content. In some implementations, the second content frame is presented for a variable duration of time. In some implementations, the diagnostic content includes a fourth content frame including EEG synchronization content, and a fifth content frame indicating whether the user earned a reward, the fifth content frame sequentially following the fourth content frame. In some implementations, the fourth content frame is presented for a fixed duration of time. In some implementations, the EEG synchronization content of the fourth content frame configured to build anticipation in the user. Some implementations include applying a tag to the EEG signals to indicate a time in the EEG signals that correlates to a transition from the second content frame to the third content frame. Some implementations include applying a tag to the EEG signals to indicate a time in the EEG signals that correlates to a transition from the fourth content frame to the fifth content frame.

Particular implementations of the subject matter described in this specification can be implemented so as to realize one or more of the following technical advantages. Implementations synchronize the presentation of diagnostic content with measurement of psychophysiological data in a manner may reduce noise in the measured signals.

The details of one or more implementations of the subject matter of this disclosure are set forth in the accompanying drawings and the description below. Other features, aspects, and advantages of the subject matter will become apparent from the description, the drawings, and the claims.

### DESCRIPTION OF DRAWINGS

FIG. 1 depicts block diagram of an example electroencephalogram (EEG) measurement system in accordance with implementations of the present disclosure.

FIG. 2 depicts an exemplary series of diagnostic content frames in accordance with implementations of the present disclosure.

FIG. 3 depicts a flowchart of an example process for synchronizing electroencephalogram (EEG) measurements with presentation of diagnostic content in accordance with implementations of the present disclosure.

FIG. 4 depicts a schematic diagram of a computer system that may be applied to any of the computer-implemented methods and other techniques described herein.

Like reference numbers and designations in the various drawings indicate like elements.

### DETAILED DESCRIPTION

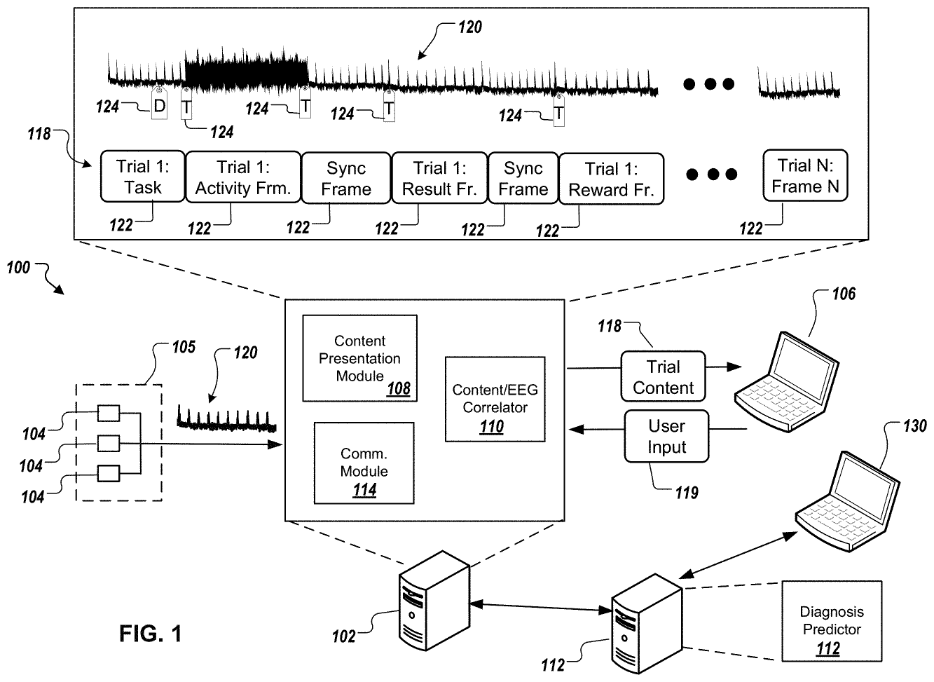

FIG. 1 depicts a block diagram of an example electroencephalogram (EEG) measurement system 100. The system includes an EEG synchronization module 102 configured to synchronizing EEG measurements with presentation of diagnostic content 118. The EEG synchronization module 102 is in communication with brainwave sensors 104, a content presentation system 106, and, optionally, one or more user computing devices 130. The EEG synchronization module 102 can be implemented in hardware or software. For example, the EEG synchronization module 102 can be a hardware or a software module that is incorporated into a computing system such as a server system (e.g., a cloud-based server system), a desktop or laptop computer, or a mobile device (e.g., a tablet computer or smartphone). The EEG synchronization module 102 includes several sub-modules which are described in more detail below. That is, some or all of the functions of the EEG synchronization module 102 (or its sub-modules) can be provided as a block of code, which upon execution by a processor, causes the processor to perform functions described below. Some or all of the functions of EEG synchronization module 102 (or its sub-modules) can be implemented in electronic circuitry, e.g., as field programmable gate array (FPGA) or an application specific integrated circuit (ASIC).

As a whole, the EEG measurement system 100 presents diagnostic content 118 to a patient while the patient's brain electrical activity is measured through an EEG sensor system 105. The EEG measurement system 100 synchronizes the EEG measurements 120 with the presentation of the diagnostic content 118. For example, the sequence of the content, timing of the presentation of the content, type of content presented, or a combination thereof can be selected in order to synchronize EEG measurement data 120 with the diagnostic content 118 being presented. That is, the EEG synchronization system 100 can intersperse particular types of EEG synchronization frames within the diagnostic content 118 in order to appropriately prepare the patient for a subsequent stimulus contained in the diagnostic content 118 so that EEG measurements of the patient's response to the stimulus is appropriately synchronized with the content.

As discussed in more detail below, in one example, EEG synchronization frames can be used to calm a patient following diagnostic content that requires the patient to perform an active task to e.g., obtain baseline EEG measurement data prior to presenting a different stimulus to the patient. In other words, the EEG synchronization system can employ an EEG synchronization frame to remove noise caused by the patient's physiological movement prior to presenting a stimulus intended to trigger a mental response by the user. In another example, EEG synchronization frames can be used to train a patient's focus prior to presenting a stimulus to the patient in the diagnostic content.

While the present disclosure is described in the context of an EEG synchronization system, it is understood that the techniques and processes described herein are applicable outside of this context. For example, the techniques and processes described herein may be applicable to other types of psychophysiological measurement systems including, but not limited to, magnetoencephalography (MEG) event-related optical signal (EROS), pupillography, or galvanic skin response (GSR).

The EEG synchronization module 102 coordinates the presentation of diagnostic content 118 to a patient during measurement of the patient's brainwaves (e.g., EEG measurement). The EEG synchronization module 102 provides diagnostic content 118 that synchronizes EEG measurements of the patient's brainwaves to the presentation of the content. The EEG synchronization module 102 provides the diagnostic content 118 to the content presentation system 106 for presentation to the patient. During the presentation of the diagnostic content 118 to the patient, the EEG synchronization module 102 obtains EEG signals 120 representing the patient's brainwaves from an EEG sensor system 105. In some examples, the EEG synchronization module 102 can send the brainwave signals to a diagnosis prediction system 112 to identify stimulus response patterns that are indicative of a future risk of, e.g., psychological conditions such as depression. As discussed below, the diagnosis prediction system 112 can employ a machine learning model trained on clinical test data sets to predict a patient's future likelihood of experiencing a psychological condition. The diagnosis prediction system 112 can provide a binary output or probabilistic output (e.g., a risk score) indicating the likelihood that the patient's will experience depression over a predefined period of time. The diagnosis prediction system 112 sends the output data to a computing device 130 associated with the patient's doctor (e.g., a psychiatrist), the doctor's office computer or mobile device.

In general, any sensors capable of detecting brainwaves may be used. For example, the brainwave sensors 104 can be one or more individual electrodes (e.g., multiple EEG electrodes) that are connected to the EEG synchronization module 102 by wired connection. The brainwave sensors 104 can be part of a brainwave sensor system 105 that is in communication with the EEG synchronization module 102. A brainwave sensor system 105 can include multiple individual brainwave sensors 104 and computer hardware (e.g., processors and memory) to receive, process, and/or display data received from the brainwave sensors 104. Example brainwave sensor systems 105 can include, but are not limited to, EEG systems, a wearable brainwave detection device, a magnetoencephalography (MEG) system, and an Event-Related Optical Signal (EROS) system, sometimes also referred to as “Fast NIRS” (Near Infrared spectroscopy). A brainwave sensor system 105 can transmit brainwave data to the EEG synchronization module 102 through a wired or wireless connection.

The content presentation system 106 is configured to present content 118 to the patient for each diagnostic trial while the patient's brainwaves are measured during the diagnostic testing. For example, the content presentation system 106 can be a multimedia device, such as a desktop computer, a laptop computer, a tablet computer, or another multimedia device that is in electronic communication with the EEG synchronization module 102. Further, the content presentation system 106 can receive input from the patient and provide the patient input 119 back to the EEG synchronization module 102.

The EEG synchronization module 102 obtains EEG data of a patient's brainwaves while the patient is presented with diagnostic content that is designed to trigger responses in particular brain systems, e.g., a brain system related to depression. During a diagnostic test, for example, a patient may be presented with diagnostic content during several trials. Each trial can include diagnostic content with stimuli designed to trigger responses in one particular brain system or multiple different brain systems. As one example a trial could include diagnostic content with physically active tasks for a patient to perform in order to achieve a reward so as to stimulate the dopaminergic reward system in the brain.

The EEG synchronization module 102 includes several sub-modules, each of which can be implemented in hardware or software. The EEG synchronization module 102 includes a content presentation module 108, a stimulus/EEG correlator 110, and a communication module 114. The EEG synchronization module 102 can be implemented as a software application executed by computing device 106. In some implementations, the sub-modules can be implemented on different computing devices. For example, one or both of the content presentation module 108 and stimulus/EEG correlator 110 can be implemented on the content presentation systems 106 with one or both of the stimulus/EEG correlator 110 and the machine learning model 112 being implemented on a server system (e.g., a cloud server system).

The communication module 114 provides a communication interface for the EEG synchronization module 102 with the brainwave sensors 104. The communication module 114 can be a wired communication (e.g., USB, Ethernet, fiber optic), wireless communication module (Bluetooth, ZigBee, WiFi, infrared (IR)). The communication module 114 can serve as an interface with other computing devices, the content presentation system 106, diagnosis prediction system 112, and user computing devices 130. The communication module 114 can be used to communicate directly or indirectly, through a network, with the brainwave sensor system 105, the content presentation system 106, user computing devices 130, or a combination thereof.

The content presentation module 108 controls the presentation of diagnostic content on the content presentation system 106. The content presentation module 108 can select diagnostic content for presentation to a patient in order to diagnose a potential mental health condition of the patient. For example, the content presentation module 108 can provide diagnostic content that synchronizes the EEG signals 120 measured from the patient with the presentation of the content itself.

FIG. 1 illustrates an example series of diagnostic content 118 that is configured to synchronize EEG signals 120 with the presentation of the content. For example the diagnostic content 118 includes a series of diagnostic content frames 122. The series includes, for example, a series of different types of diagnostic content frames. Diagnostic content frames 122 can include, but are not limited to, task frames, activity frames, EEG synchronization frames, and stimulus frames. For example, a task frame may provide a patient with information about a particular task that the participant is requested to complete. In some examples, a task frame may include information about a possible reward the patient may earn for completing the task. An activity frame may provide the patient with graphics for media indicating the status of the task, or encouraging patient to complete the task. In other words, an activity frame is generally presented to the patient while the patient is performing the task described in the task frame. Stimulus frames, such as result frame and reward frame shown in FIG. 1, include images, information, video media, or audio intended to provoke a mental response from a patient.

EEG synchronization frames, for example, can present content that is specifically configured to produce a change in the EEG signals measured from the user. In some instances, EEG synchronization frames can be used to remove artifacts of a patient's response to a previous stimulus or activity EEG signals from EEG signals 120 (e.g., to re-establish a baseline EEG signal). For example, an EEG synchronization frame can be presented after a diagnostic content frame, such as an activity frame, that requires a patient to perform a significant amount of physiological activity such as movement. Such EEG synchronization frames can be used to calm the patient's motor activity in order to establish a baseline EEG signal. In other words, the EEG synchronization frame can serve as a kind of pre-filter to clean excessive noise out of the patient's EEG signal following a period of significant motor activity and prior to presenting a new diagnostic stimulus to the patient.

In some instances, EEG synchronization frames can be used to prepare a patient's EEG signal measurements for a subsequent diagnostic stimulus or activity. For example, an EEG synchronization frame can be used to prepare the patient's mental activity for the presentation of a subsequent stimulus. That is, an EEG synchronization frame can be presented immediately prior to a particular diagnostic stimulus in order to prepare the patient for the upcoming stimulus frame. As one example, an EEG synchronization frame can be used to build anticipation in a patient prior to presenting a particular type of stimulus frame.

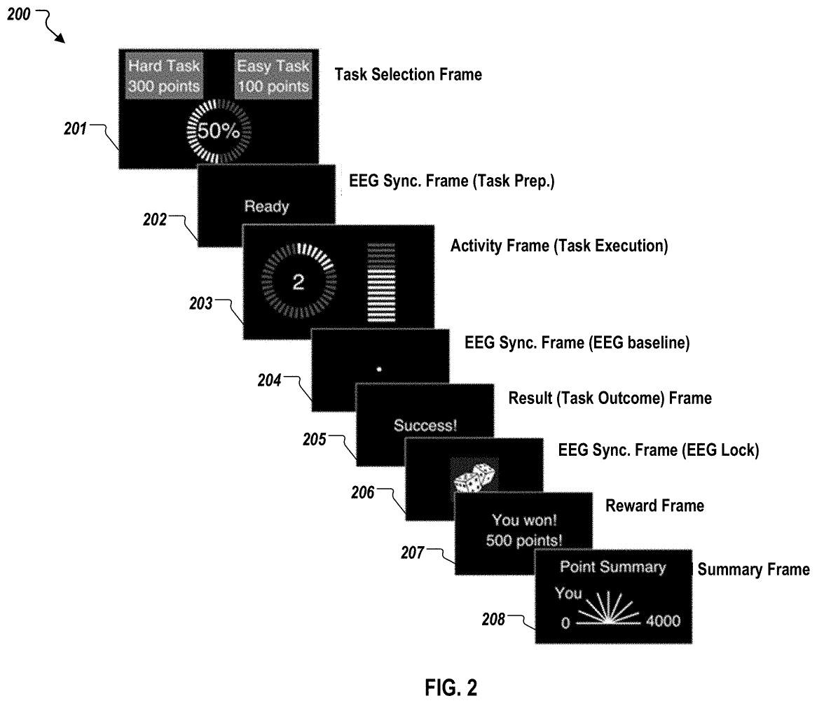

FIG. 2 depicts an exemplary series 200 of diagnostic content frames in accordance with implementations of the present disclosure. In particular, FIG. 2 illustrates diagnostic content frames of a “hot button task” that can be used as one of multiple trials in a diagnostic test. Referring to FIGS. 1 and 2, in the diagnostic content series, the first frame 201 is a task selection frame. The task selection frame presents the patient with a choice between two types of tasks; a hard task and an easy task. In addition, the task selection frame informs the patient that she may receive 300 points for completion of the hard task and 100 points for completion of the easy task. The task selection frame also provides the user with a percent likelihood of receiving a reward if successful at the task. For instance, in the example task selection frame shown the patient has a 50% chance of receiving a reward if they successfully complete either the hard or easy task.

The second frame 202 represents an EEG synchronization frame used to prepare the patient for performing the selected task. For example, the second frame may be presented for a fixed period of time (e.g., 500 ms) for each trial of the “hot button task.” That is, each time the second frame 202 is presented it will be presented for the same fixed duration of time.

The third frame 203 represents an activity frame. The activity frame is presented to the patient while the patient executes the task indicated in the task selection frame. For example, the activity frame is presented to the patient while the patient performs the “hot button task.” As an example, the activity frame shown is presented to the patient while the patient pushes a particular key on a keyboard for a set number of times. For example, if the patient selected the easy task they may be required to press the “L” key 30 times in seven seconds with their dominant hand. If the patient selected the hard task they may be required to press the “S” key 100 times in 21 seconds using their non-dominant hand. In the example shown, the activity frame includes an animated timer that counts the time down while the patient performs the selected task and an animated vertical status bar that is progressively illuminated to illustrate the number of times that the patient has pressed the “hot button.”

The fourth frame 204 is an EEG synchronization frame that is configured to establish an EEG baseline following the activity frame. For example, the fourth frame 204 provides a fixation point on which the patient focuses her eyes for a period of time in order to cool down and reduce motor movements following the execution of the “hot button task.” In some implementations, and EEG synchronization frame configured to establish an EEG baseline is presented for a variable period of time (e.g., 400-600 ms) for each trial of the “hot button task.” That is, each time the fourth frame 204 is presented it will be presented for a different duration of time.

The fifth frame 205 is a stimulus frame indicating the result of the task executed by the patient. For example, the fifth frame 205 indicates that the patient successfully completed their selected task. In some implementations, as discussed below, the content/EEG correlator 110 applies a tag 124 to the EEG signals 120 to indicate the point in time when the fourth frame 204 transitioned into the fifth frame 205 (e.g., a timestamp). For example, the applied tag 124 indicates when the patient was presented with the result frame so as to precisely measure the patient's response to their success or failure at the task. For example, the tag 124 can be a timestamp that time locks the participant's neural response to the presentation of frame 205, e.g., to permit accurate assessment of the participant's response to successfully completing the task versus failing to complete the task. For example, an ERP (Event Related Potential) component called the reward positivity can be extracted during this time window, which in the past has been shown to relate to self-reported levels of anhedonia and reward responsiveness.

The sixth frame 206 represents an EEG synchronization frame that is used to prepare the patient for the next diagnostic stimulus that will be presented in the seventh frame 207. For example, the sixth frame 206 represents an EEG synchronization frame that is configured to focus the patient's measured EEG signals in order to evaluate the patient's anticipation of possibly receiving a reward. For example, the sixth frame 206 can include content such as an image, video, and/or audio intended to build anticipation. In some examples, can EEG synchronization frame that is used to build anticipation in the patient is presented for a fixed period of time (e.g., 200 ms) before changing to a subsequent frame. For example, in order to build anticipation in a patient and measure an effect of the anticipation on the patient's brain waves (represented by the patient's EEG signals), the patient's brain waves can be substantially synchronized with the timing of the EEG synchronization frame 206 by always presenting the frame for the same duration of time during each trial of the diagnostic test. That is, each time the sixth frame 206 is presented it will be presented for the same fixed duration of time.

The seventh frame 207 is a stimulus frame indicating whether the patient will receive a reward. For example, the seventh frame 207 indicates that the patient won 500 points. In some implementations, as discussed below, the content/EEG correlator 110 applies a tag 124 to the EEG signals 120 to indicate the point in time when the sixth frame 206 transitioned into the seventh frame 207. For example, the applied tag 124 indicates when the patient was presented with the reward frame so as to precisely measure the patient's response to receiving (or not receiving) a reward.

The eighth frame 208 represents an optional summary frame. For example, the summary frame indicates a summary of the total number of points that the patient has received throughout the entire diagnostic test so far.

The content presentation module 108 can send data related to the content presented on the content presentation system 106 to the content/EEG correlator 110. For example, the stimulus/EEG correlator 110 receives the EEG data from the brainwave sensors 104 and the content data from the content presentation module 108. The stimulus/EEG correlator 110 can correlate the timing of the content presentation to the patient with the EEG data. The content/EEG correlator 110 can add data tags 124 to the EEG signals 120 to indicate, for example, transitions between different types of diagnostic content (e.g., transitions between content frames). For example, the tags 124 can include timestamps indicating a start and stop time of when the content was presented. In some examples, a tag 124 can include a label indicating the type of content. For example, a tag 124 can indicate a value of the content such as whether the content was positive (winning points) or negative (a failed task).

In some implementations, the stimulus/EEG correlator 110 can add tags 124 that indicate a decision made by the patient in response to a diagnostic content frame. For example, the tag 124 labeled “D” in FIG. 1 can include data representing which task the patient selected when presented with a task selection content frame; the “hard task” or the “easy task.”

In some examples, the stimulus/EEG correlator 110 can send tagged brainwave signals where the tags provide information including, but not limited to, an indication of the type of content presented when the brainwaves were measured, and an indication of where in the EEG signal 120 the content presentation started. That is, the tags 124 can indicate on the EEG signal 120 when a new frame of diagnostic content was presented to the patient.

The diagnosis predictor 112 determines a likelihood that the patient is experiencing or will experience a mental health condition, e.g., depression or anxiety. For example, the diagnosis predictor 112 can include a machine learning model that analyzes brainwave signals associated with one or more brain systems to determine the likelihood that the patient will experience a type of depression, major depressive disorder or post-partum depression, in the future. In some implementations, the diagnosis predictor 112 analyzes brainwave signals associated with one or more brain systems to determine the likelihood that the patient will experience anxiety in the future. For example, the diagnosis predictor 112 can analyze brainwaves associated with brain systems that are predictive of a particular mental health condition.

The diagnosis predictor 112 incorporates a machine learning model to identify patterns in the brainwaves associated with the particular brain systems that are predictive of future depression. For example, the machine learning model can include a machine learning model that has been trained to receive model inputs, detection signal data, and to generate a predicted output, a prediction of the likelihood that the patient will experience depression in the future. In some implementations, the machine learning model is a deep learning model that employs multiple layers of models to generate an output for a received input. A deep neural network is a deep machine learning model that includes an output layer and one or more hidden layers that each apply a non-linear transformation to a received input to generate an output. In some cases, the neural network may be a recurrent neural network. A recurrent neural network is a neural network that receives an input sequence and generates an output sequence from the input sequence. In particular, a recurrent neural network uses some or all of the internal state of the network after processing a previous input in the input sequence to generate an output from the current input in the input sequence. In some other implementations, the machine learning model is a convolutional neural network. In some implementations, the machine learning model is an ensemble of models that may include all or a subset of the architectures described above.

In some implementations, the machine learning model can be a feedforward autoencoder neural network. For example, the machine learning model can be a three-layer autoencoder neural network. The machine learning model may include an input layer, a hidden layer, and an output layer. In some implementations, the neural network has no recurrent connections between layers. Each layer of the neural network may be fully connected to the next, there may be no pruning between the layers. The neural network may include an ADAM optimizer, or any other multi-dimensional optimizer, for training the network and computing updated layer weights. In some implementations, the neural network may apply a mathematical transformation, such as a convolutional transformation, to input data prior to feeding the input data to the network.

In some implementations, the machine learning model can be a supervised model. For example, for each input provided to the model during training, the machine learning model can be instructed as to what the correct output should be. The machine learning model can use batch training, training on a subset of examples before each adjustment, instead of the entire available set of examples. This may improve the efficiency of training the model and may improve the generalizability of the model. The machine learning model may use folded cross-validation. For example, some fraction (the “fold”) of the data available for training can be left out of training and used in a later testing phase to confirm how well the model generalizes. In some implementations, the machine learning model may be an unsupervised model. For example, the model may adjust itself based on mathematical distances between examples rather than based on feedback on its performance.

A machine learning model can be trained to recognize brainwave patterns that indicate a patient's potential risk of one or more types of mental health conditions. For example, the machine learning model can correlate identified brainwaves from particular brain system(s) with patterns that are indicative of those leading to a type of depression such as major depressive disorder or post-partum depression. In some examples, the machine learning model can be trained on clinical study data sets based on actual diagnoses of mental health conditions. The machine learning model can be trained to identify brainwave signal patterns from relevant brain systems that occur prior to the onset of depression. In some implementations, the machine learning model can refine the ability to predict depression from brainwaves associated brain systems such as those described herein. For example, the machine learning model can continue to be trained on data from actual diagnoses of previously monitored patients that either confirm or correct prior predictions of the model or on additional clinical trial data.

In some examples, the diagnosis predictor 112 can provide a binary output, a yes or no indication of whether the patient is likely to experience depression or anxiety. In some examples, the diagnosis predictor 112 provides a risk score indicating a likelihood that the patient will experience a mental health condition (e.g., a score from 0-10 or a percentage indicating a probability that the patient will experience depression or anxiety). In some implementations, the diagnosis predictor 112 can output annotated brainwave graphs. For example, the annotated brainwave graphs can identify particular brainwave patterns that are indicative of future mental health conditions.

In some implementations, the diagnosis predictor 112 sends output data indicating the patient's likelihood of experiencing depression to a user computing device 130. For example, the diagnosis predictor 112 can send the output of the machine learning model to a user computing device 130 associated with the patient's doctor.

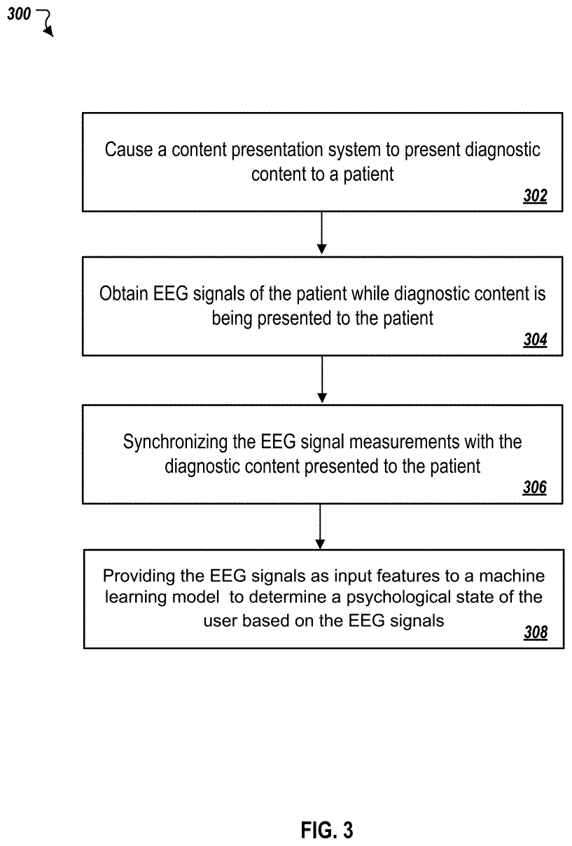

FIG. 3 depicts a flowchart of an example process 300 for synchronizing EEG measurements with presentation of diagnostic content. In some implementations, the process 300 can be provided as one or more computer-executable programs executed using one or more computing devices. In some examples, the process 300 is executed by a system such as EEG synchronization module 102 of FIG. 1. In some implementations, all or portions of process 300 can be performed on a local computing device, a desktop computer, a laptop computer, or a tablet computer. In some implementations, all or portions of process 300 can be performed on a remote computing device, a server system, a cloud-based server system.

The system causes a content presentation system to present diagnostic content to a patient (302). For example, the system provides diagnostic content to a user interface system (e.g., a computer system) for display to a patient. As discussed above, the diagnostic content can include a diagnostic test made of multiple sets of trial content. Each set of trial content can include a series of diagnostic content frames (e.g., as shown in FIG. 2). The system obtains EEG signals of the patient while the diagnostic content is being presented to the patient (304). For example, the system can measure the patient's brainwaves using an EEG sensor system while the diagnostic content is presented to the patient. The system synchronizes the EEG signal measurements with the diagnostic content presented to the patient (306). For example, the system can include EEG synchronization frames within the diagnostic content to synchronize the patients' EEG measurements with the presentation of the diagnostic content. In some examples, the system can apply data tags to the EEG signals. The data tags can be used to indicate information including, but not limited to, the timing of transitions between content frames in relation to the EEG measurements, the type of content presented, choices made by the patient, or a combination thereof. The system provides the EEG signals as input features to a machine learning model to determine a psychological and/or motivational state of the user based on the EEG signals (308). For example, the system can provide the EEG signals alone or in combination with data tags as input features to a diagnostic machine learning model in order to determine whether the patient is or will experience a mental health condition.

Further to the descriptions above, a patient may be provided with controls allowing the user to make an election as to both if and when systems, programs, or features described herein may enable collection of user information. In addition, certain data may be treated in one or more ways before it is stored or used, so that personally identifiable information is removed. For example, a patient's identity may be treated so that no personally identifiable information can be determined for the patient, or a patient's test data and/or diagnosis cannot be identified as being associated with the patient. Thus, the patient may have control over what information is collected about the patient and how that information is used.

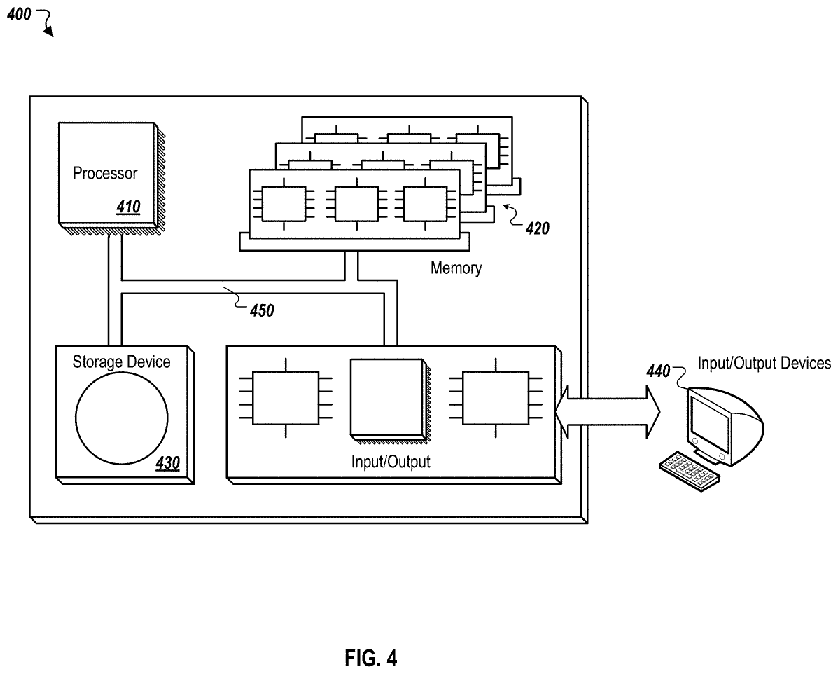

FIG. 4 is a schematic diagram of a computer system 400. The system 400 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 400) and their structural equivalents, or in combinations of one or more of them. The system 400 is intended to include various forms of digital computers, such as laptops, desktops, workstations, personal digital assistants, servers, blade servers, mainframes, and other appropriate computers, including vehicles installed on base units or pod units of modular vehicles. The system 400 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 400 includes a processor 410, a memory 420, a storage device 430, and an input/output device 440. Each of the components 410, 420, 430, and 440 are interconnected using a system bus 450. The processor 410 is capable of processing instructions for execution within the system 400. The processor may be designed using any of a number of architectures. For example, the processor 410 may be a CISC (Complex Instruction Set Computers) processor, a RISC (Reduced Instruction Set Computer) processor, or a MISC (Minimal Instruction Set Computer) processor.

In one implementation, the processor 410 is a single-threaded processor. In another implementation, the processor 410 is a multi-threaded processor. The processor 410 is capable of processing instructions stored in the memory 420 or on the storage device 430 to display graphical information for a user interface on the input/output device 440.

The memory 420 stores information within the system 400. In one implementation, the memory 420 is a computer-readable medium. In one implementation, the memory 420 is a volatile memory unit. In another implementation, the memory 420 is a non-volatile memory unit.

The storage device 430 is capable of providing mass storage for the system 400. In one implementation, the storage device 430 is a computer-readable medium. In various different implementations, the storage device 430 may be a floppy disk device, a hard disk device, an optical disk device, or a tape device.

The input/output device 440 provides input/output operations for the system 400. In one implementation, the input/output device 440 includes a keyboard and/or pointing device. In another implementation, the input/output device 440 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

While the present disclosure is described in the context of a psychological diagnostic system, it is understood that the techniques and processes described herein are applicable outside of this context. For example, the techniques and processes described herein may be applicable to other types of diagnostic machine learning systems including, but not limited to, medical diagnostic systems, computer software diagnostic (debugging) systems, computer hardware diagnostic systems, or quality assurance (e.g., in manufacturing) diagnostic systems.

1. A computer-implemented electroencephalogram (EEG) measurement method for synchronizing EEG measurements with presentation of diagnostic content, the method executed by one or more processors and comprising:
causing, by the one or more processors, a presentation system to present diagnostic content a user, the diagnostic content comprising:
a first content frame prompting the user to perform a physical task,
a second content frame comprising EEG synchronization content, the second content frame sequentially following the first content frame, and
a third content frame indicating an outcome of the physical task, the third content frame sequentially following the second content frame;
obtaining, by the one or more processors and from a brainwave sensor, EEG signals of the user during presentation of the diagnostic content; and
providing, by the one or more processors, the EEG signals as input features to a machine learning model that is trained to predict a psychological state of the user based on the EEG signals.

2. The method of claim 1, wherein the second content frame comprises EEG synchronization content configured to establish a baseline EEG signal.

3. The method of claim 2, wherein the EEG synchronization content includes fixation content.

4. The method of claim 1, wherein the second content frame is presented for a variable duration of time.

5. The method of claim 1, wherein the diagnostic content comprises:
a fourth content frame comprising EEG synchronization content; and
a fifth content frame indicating whether the user earned a reward, the fifth content frame sequentially following the fourth content frame.

6. The method of claim 5, wherein the fourth content frame is presented for a fixed duration of time.

7. The method of claim 5, wherein the EEG synchronization content of the fourth content frame configured to build anticipation in the user.

8. The method of claim 1, further comprising applying a tag to the EEG signals to indicate a time in the EEG signals that correlates to a transition from the second content frame to the third content frame.

9. The method of claim 5, further comprising applying a tag to the EEG signals to indicate a time in the EEG signals that correlates to a transition from the fourth content frame to the fifth content frame.

10. The method of claim 1, wherein the diagnostic content comprises:
a fourth content frame comprising EEG synchronization content; and
a fifth content frame indicating whether the user earned a reward, the fifth content frame sequentially following the fourth content frame, and
wherein the method further comprises:
applying a first tag to the EEG signals to indicate a time in the EEG signals that correlates to a transition from the second content frame to the third content frame; and
applying a second tag to the EEG signals to indicate a time in the EEG signals that correlates to a transition from the fourth content frame to the fifth content frame.

11. An electroencephalogram (EEG) measurement system for synchronizing EEG measurements with presentation of diagnostic content, the system comprising:
one or more processors; and
one or more tangible, non-transitory media operably connectable to the one or more processors and storing instructions that, when executed, cause the one or more processors to perform operations comprising:
causing a presentation system to present diagnostic content to a user, the diagnostic content comprising:
a first content frame prompting the user to perform a physical task,
a second content frame comprising EEG synchronization content, the second content frame sequentially following the first content frame, and
a third content frame indicating an outcome of the physical task, the third content frame sequentially following the second content frame;
obtaining, from a brainwave sensor, EEG signals of the user during presentation of the diagnostic content; and
providing the EEG signals as input features to a machine learning model that is trained to predict a psychological state of the user based on the EEG signals.

12. The system of claim 11, wherein the second content frame comprises EEG synchronization content configured to establish a baseline EEG signal.

13. The system of claim 11, wherein the second content frame is presented for a variable duration of time.

14. The system of claim 11, wherein the diagnostic content comprises:
a fourth content frame comprising EEG synchronization content; and
a fifth content frame indicating whether the user earned a reward, the fifth content frame sequentially following the fourth content frame.

15. The system of claim 15, wherein the fourth content frame is presented for a fixed duration of time.

16. The system of claim 15, wherein the EEG synchronization content of the fourth content frame configured to build anticipation in the user.

17. The system of claim 11, further comprising applying a tag to the EEG signals to indicate a time in the EEG signals that correlates to a transition from the second content frame to the third content frame.

18. The system of claim 15, further comprising applying a tag to the EEG signals to indicate a time in the EEG signals that correlates to a transition from the fourth content frame to the fifth content frame.

19. The method of claim 11, wherein the diagnostic content comprises:
a fourth content frame comprising EEG synchronization content; and
a fifth content frame indicating whether the user earned a reward, the fifth content frame sequentially following the fourth content frame, and
wherein the method further comprises:
applying a first tag to the EEG signals to indicate a time in the EEG signals that correlates to a transition from the second content frame to the third content frame; and
applying a second tag to the EEG signals to indicate a time in the EEG signals that correlates to a transition from the fourth content frame to the fifth content frame.

20. A non-transitory computer readable storage medium storing instructions that, when executed by at least one processor, cause the at least one processor to perform operations comprising:
causing a presentation system to present diagnostic content a user, the diagnostic content comprising:
a first content frame prompting the user to perform a physical task,
a second content frame comprising EEG synchronization content, the second content frame sequentially following the first content frame, wherein the second content frame is presented for a variable duration of time,
a third content frame indicating an outcome of the physical task, the third content frame sequentially following the second content frame,
a fourth content frame comprising EEG synchronization content, wherein the fourth content frame is presented for a fixed duration of time, and
a fifth content frame indicating whether the user earned a reward, the fifth content frame sequentially following the fourth content frame;
obtaining, by the one or more processors and from a brainwave sensor, EEG signals of the user during presentation of the diagnostic content;
applying a first tag to the EEG signals to indicate a time in the EEG signals that correlates to a transition from the second content frame to the third content frame;
applying a second tag to the EEG signals to indicate a time in the EEG signals that correlates to a transition from the fourth content frame to the fifth content frame; and
providing the EEG signals, the first tag, and the second tag as input features to a machine learning model that is trained to predict a psychological state of the user based on the EEG signals.
